(19) United States
(12) Patent Application Publication
(10) Pub. No.: US 2007/0179613 A1
(43) Pub. Date: Aug. 2, 2007

(54) Passive lubricating prosthetic joint

(75) Inventor: Eric S. Heinz, Memphis, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/343,080

(22) Filed: Jan. 30, 2006

(51) Int. Cl. A61F 2/44; A61F 2/30; A61F 2/38; A61F 2/32

(52) U.S. Cl. 623/17.12; 623/17.15; 623/20.31; 623/22.13; 623/23.39

(57) ABSTRACT

Prosthetic joints, intervertebral prosthetic implants and methods of replacing intervertebral discs are provided. In an exemplary embodiment, a intervertebral prosthetic implant can include a pair of substantially rigid members and an articulation member having an articulation surface and configured to facilitate motion between the first and second members. The first member can be provided with at least one capillary channel configured to transport lubricious fluid proximate the articulation surface of the articulation member.

### BACKGROUND

1. Field of the Disclosure

The present disclosure relates to the field of orthopedic surgery and has particular application to a total artificial joint and its post implantation performance.

2. Description of Related Art

Lubrication of a natural joint is a complex process that allows the joint to operate under a variety of conditions. Such conditions can include: maximum joint surface velocity and sudden and prolonged applied load. Articular cartilage is filled with synovial fluid that is squeezed from the surface upon loading. At high loads and low velocity, boundary lubrication is present as a means to protect the surfaces and minimize their contact. At higher velocities, fluid film lubrication is generated between the surfaces because of pressure build-up, which substantially completely separates the surfaces. Since cartilage is highly deformable under pressure, this deformation can enhance the thickness of the fluid film, a process which is typically termed elastohydrodynamic lubrication. Synovial fluid can act as a shock absorber, particularly under high loads when the synovial fluid's molecules undergo conformational changes. The energy of conformation is stored and released later. In contrast, the molecules serve as lubricants at low loads because they are flexible enough to maintain their conformation.

People who suffer from the pain and mobility loss associated with diseased joints may benefit from implants designed to improve their situation. Orthopedic implants made from metals, alloys, polymers, polymer blends and metal/polymer blends may alleviate the decreased motion in these diseased joints. Biocompatibility and bioresorbability of a material are often significant criteria for a successful implant. Wear occurring at the interface of surfaces within the joint can be a significant contributor to joint failure as well as to deleterious effects in collateral systems resulting from wear debris. The long-term performance of traditional prosthetic joints has suffered from, among other things, a lack of an effective, long-term lubrication mechanism, whether by effectively delivering a synovial fluid substitute or by replicating the delivery of natural fluids to joint articulation surfaces.

### SUMMARY

Accordingly, the present disclosure is directed to various embodiments of a passive lubricating intervertebral prosthetic implant, a prosthetic joint and a method of replacing intervertebral discs. In an exemplary embodiment an intervertebral prosthetic implant includes a pair of substantially rigid members configured to engage adjacent vertebrae and an articulation member configured to facilitate motion between the members. One of the members has at least one capillary channel configured to transport lubricious fluid proximate the articulation surface of the articulation member at least in part by capillary force.

In another exemplary embodiment, an intervertebral prosthetic implant includes a pair of substantially rigid members configured to engage adjacent vertebrae and an articulation member configured to facilitate motion between the members. One of the members has at least one fluid channel configured to allow lubricious fluid to flow proximate the articulation surface of the articulation member. A semi-permeable membrane is disposed proximate to the proximal end of at least one of the fluid channels.

In another exemplary embodiment, an intervertebral prosthetic implant includes a pair of substantially rigid members configured to engage adjacent vertebrae. One of the members includes an articulation member and at least one capillary channel configured to transport lubricious fluid to the articulation surface of the articulation member at least in part by capillary force.

In another exemplary embodiment, an intervertebral prosthetic implant includes a pair of substantially rigid members configured to engage adjacent vertebrae. One of the members includes an articulation member and at least one fluid channel configured to allow lubricious fluid to flow proximate the articulation surface of the articulation member. A semi-permeable membrane is disposed proximate to the proximal end of at least one of the fluid channels.

In another exemplary embodiment, a prosthetic joint includes a pair of substantially rigid members configured to engage first and second bones and an articulation member configured to facilitate motion between the pair of substantially rigid members. At least one of the substantially rigid members has one or more capillary channels configured to transport lubricious fluid to the articulation surface of the articulation member at least in part by capillary force.

In another exemplary embodiment, a prosthetic joint includes a pair of substantially rigid members configured to engage first and second bones and an articulation member configured to facilitate motion between the pair of substantially rigid members. At least one of the substantially rigid members has one or more fluid channels configured to allow lubricious fluid to flow proximate the articulation surface of the articulation member. A semi-permeable membrane is disposed proximate to the proximal end of at least one of the fluid channels.

In another exemplary embodiment, a method of replacing at least a portion of an intervertebral disc includes the steps of gaining access to the intervertebral disc; removing at least a portion of the intervertebral disc to create an intervertebral space; and inserting a prosthetic disc into the intervertebral space. The prosthetic disc includes a pair of substantially rigid members configured to engage adjacent vertebrae and an articulation member configured to facilitate motion between the members. At least one of the members has one or more capillary channels configured to transport lubricious fluid to the articulation surface of the articulation member at least in part by capillary force.

In another exemplary embodiment, a method of replacing at least a portion of an intervertebral disc includes the steps of gaining access to the intervertebral disc; removing at least a portion of the intervertebral disc to create an intervertebral space; and inserting a prosthetic disc into the intervertebral space. The prosthetic disc includes a pair of substantially rigid members configured to engage adjacent vertebrae and an articulation member configured to facilitate motion between the members. At least one of the members has one or more fluid channel configured to allow lubricious fluid to flow proximate the articulation surface of the articulation member. A semi-permeable membrane is disposed proximate the proximal end of at least one of the fluid channels and the articulation surface of the articulation member.

In another exemplary embodiment, an intervertebral prosthetic implant includes a pair of substantially rigid members configured to engage adjacent vertebrae and an articulation member configured to facilitate motion between the members. One of the members has at least one fluid channel configured to allow lubricious fluid to flow proximate the articulation surface of the articulation member. A semi-permeable membrane is disposed between the proximal end of at least one of the fluid channels and the articulation surface of the articulation member.

### BRIEF DESCRIPTION OF THE DRAWINGS

The present disclosure may be better understood, and its numerous features and advantages made apparent to those skilled in the art by referencing the accompanying drawings.

FIG. 1 is a lateral view of a portion of a vertebral column.

FIG. 2 is a lateral view of a pair of adjacent vertebrae.

FIG. 3 is a top plan view of a vertebra.

FIG. 4 is a plan view of a component of an intervertebral prosthetic implant.

FIG. 5 is a plan view of a component of an intervertebral prosthetic implant.

FIG. 6 is an anterior view of an intervertebral prosthetic implant.

FIG. 7 is an exploded anterior view of an intervertebral prosthetic implant.

FIG. 8 is a lateral view of an intervertebral prosthetic implant.

FIG. 9 is an exploded lateral view of an intervertebral prosthetic implant.

FIG. 10 is an exploded lateral view of an intervertebral prosthetic implant installed within an intervertebral space between a pair of adjacent vertebrae.

FIG. 11 is a plan view of a component of an intervertebral prosthetic implant.

FIG. 12 is a plan view of a component of an intervertebral prosthetic implant.

FIG. 13 is an anterior view of an intervertebral prosthetic implant.

FIG. 14 is an exploded anterior view of an intervertebral prosthetic implant.

FIG. 15 is an exploded perspective view of an intervertebral prosthetic implant.

FIG. 16 is a plan view of a component of an intervertebral prosthetic implant.

FIG. 17 is a plan view of a component of an intervertebral prosthetic implant.

FIG. 18 is a plan view of a component of an intervertebral prosthetic implant.

FIG. 19 is a plan view of a component of an intervertebral prosthetic implant.

FIG. 20 is an exploded lateral view of prosthetic hip joint.

FIG. 21 is a lateral view of an installed prosthetic knee joint.

The use of the same reference symbols in different drawings indicates similar or identical items.

### DETAILED DESCRIPTION

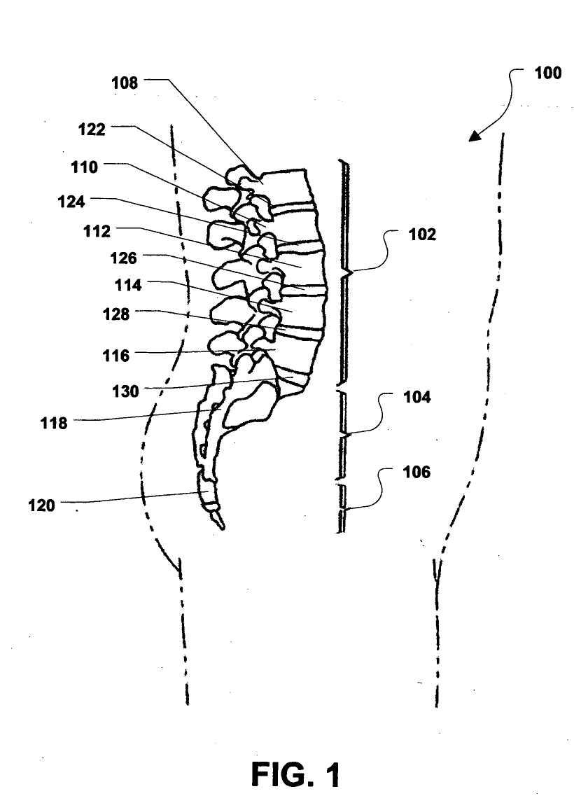

The teachings of the present application can find utility in various joint replacement situations, such as knee prosthetics, hip prosthetics and intervertebral prosthetic discs. With particular reference to intervertebral embodiments, FIG. 1 shows a portion of a vertebral column, designated 100. As depicted, the vertebral column 100 includes a lumbar region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumbar vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumbar vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of replacement, that intervertebral lumbar disc 122, 124, 126, 128, 130 can be at least partially removed and replaced with an intervertebral prosthetic disc according to one or more of the embodiments described herein. In a particular embodiment, a portion of the intervertebral lumbar disc 122, 124, 126, 128, 130 can be removed via a discectomy, or a similar surgical procedure, well known in the art. Further, removal of intervertebral lumbar disc material can result in the formation of an intervertebral space (not shown) between two adjacent lumbar vertebrae.

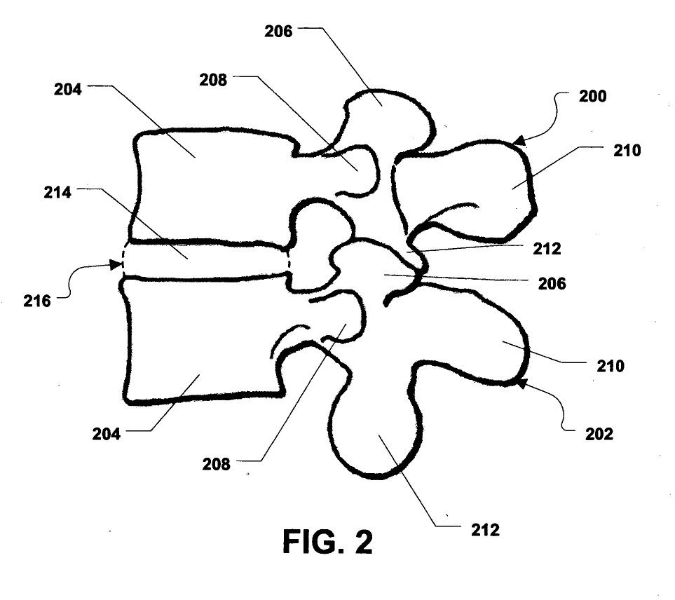

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines). As described in greater detail below, an intervertebral prosthetic disc according to one or more of the embodiments described herein can be installed within the intervertebral space 212 between the superior vertebra 200 and the inferior vertebra 202.

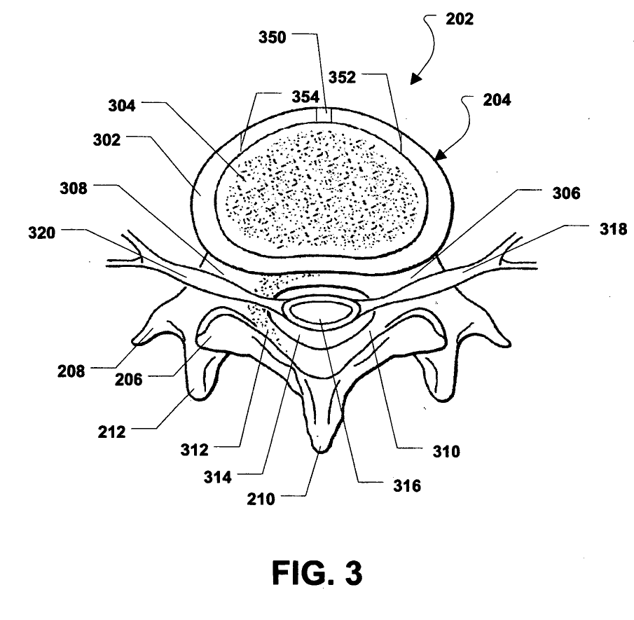

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

The vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

FIG. 3 further depicts a keel groove 350 that can be established within the cortical rim 302 of the inferior vertebra 202. Further, a first corner cut 352 and a second corner cut 354 can be established within the cortical rim 302 of the inferior vertebra 202. In a particular embodiment, the keel groove 350 and the corner cuts 352, 354 can be established during surgery to install an intervertebral prosthetic disc according to one or more of the embodiments described herein. The keel groove 350 can be established using a keel-cutting device, e.g., a keel chisel designed to cut a groove in a vertebra, prior to the installation of the intervertebral prosthetic disc. Further, the keel groove 350 is sized and shaped to receive and engage a keel, described below, that extends from an intervertebral prosthetic disc according to one or more of the embodiments described herein. The keel groove 350 can cooperate with a keel to facilitate proper alignment of an intervertebral prosthetic disc within an intervertebral space between an inferior vertebra and a superior vertebra.

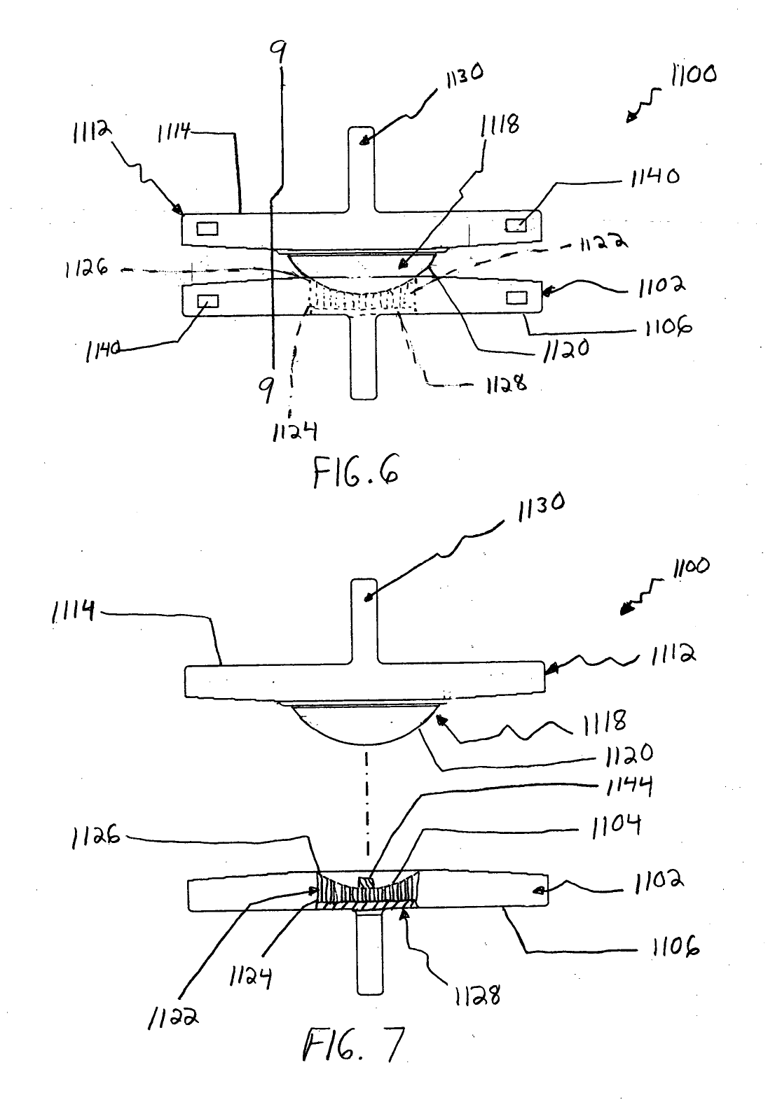

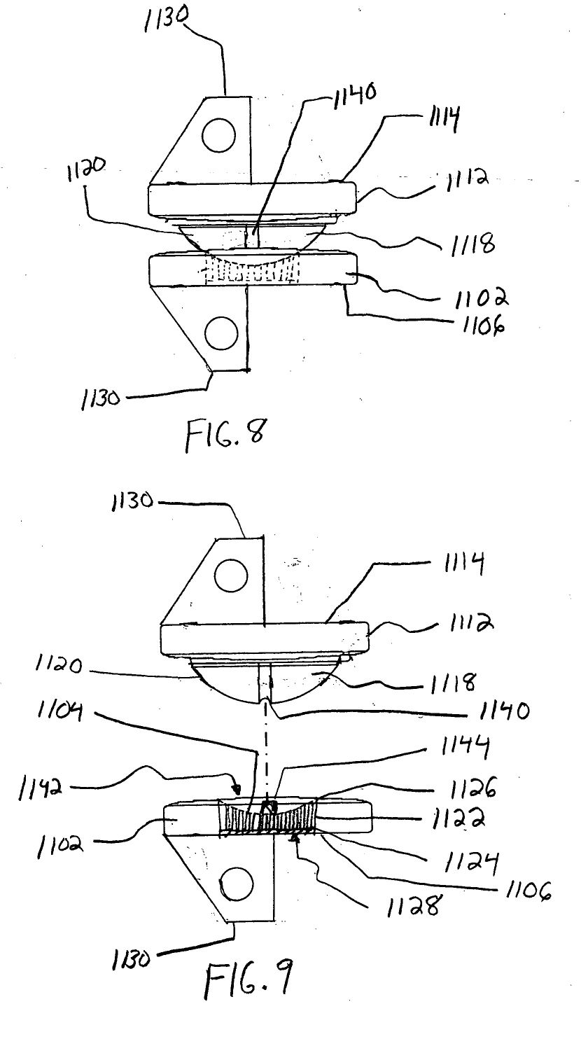

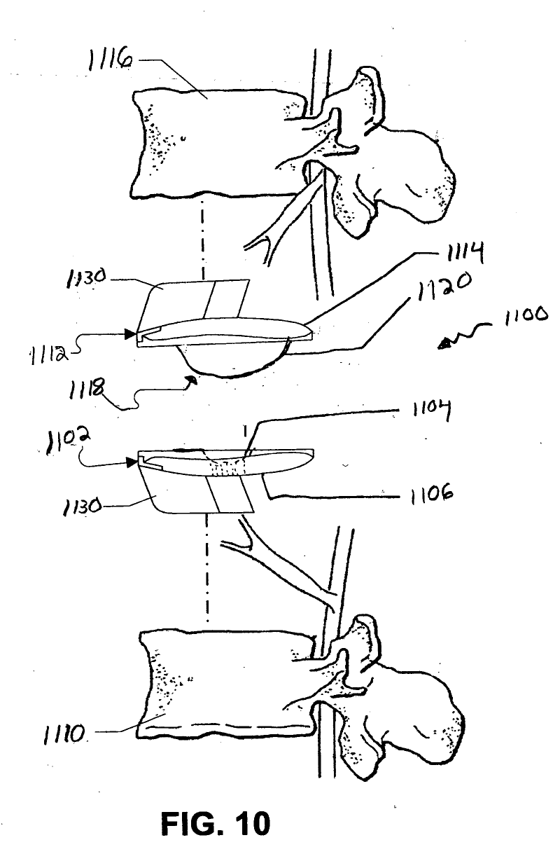

As shown in FIGS. 4-10, an exemplary embodiment is directed to an intervertebral prosthetic implant 1100 which can include a substantially rigid first member 1102 having an articulation surface 1104 and an engagement surface 1106 configured to engage a first vertebra 1110 (FIG. 10) and a substantially rigid second member 1112 having an engagement surface 1114 configured to engage a second vertebra 1116 (FIG. 10). An articulation member 1118 having an articulation surface 1120 can be disposed between the members 1102, 1112 and configured to facilitate motion between the members 1102, 1112. The first member 1102 can have at least one fluid channel 1122 (best seen in cross-sectional FIGS. 7 and 9) having a proximal end 1124 and a distal end 1126 relative to the engagement surface 1106 of the first member. The fluid channels 1122 can be configured to transport lubricious fluid proximate the articulation surface 1120 of the articulation member. A semi-permeable membrane 1128 can be disposed proximate to the proximal end 1124 of at least one of the fluid channels 1122. Alternatively, a semi-permeable membrane can be disposed at any functional position between the proximal end 1124 of at least one of the fluid channels 1122 and the articulation surface 1120 of the articulation member 1118. In various alternative embodiments, the fluid channels 1122 can be sized and shaped to act as capillary channels in transporting the lubricious fluid within the implant at least in part by capillary force as described infra.

As shown in FIGS. 4 and 8, the articulation member can include a retention groove 1140 in its articulation surface. The first member 1102 can include a retention depression 1142 and a retention projection 1144. The retention groove can be configured to receive the retention projection in order to align and substantially maintain alignment of the articulation member 1118 and the first member 1102. Alternatively, at least one of the first or second substantially rigid members 1102, 1112 can include a retention post or lip (not shown) that, under most operational conditions, does not contact the articulation surface yet prevents migration of the articulation member.

FIG. 6 through FIG. 10 show that the members 1102, 1112 can each include a keel 1130 that extends from the respective members. During installation, the keel 1130 can at least partially engage a keel slot or groove that can be established within a cortical rim of a vertebra. The keels can be sized, shaped and positioned to facilitate and maintain proper alignment of the implant. Although not shown, it will be appreciated by those skilled in the art that the keels can be angled or rotatable in order to facilitate various surgical approaches.

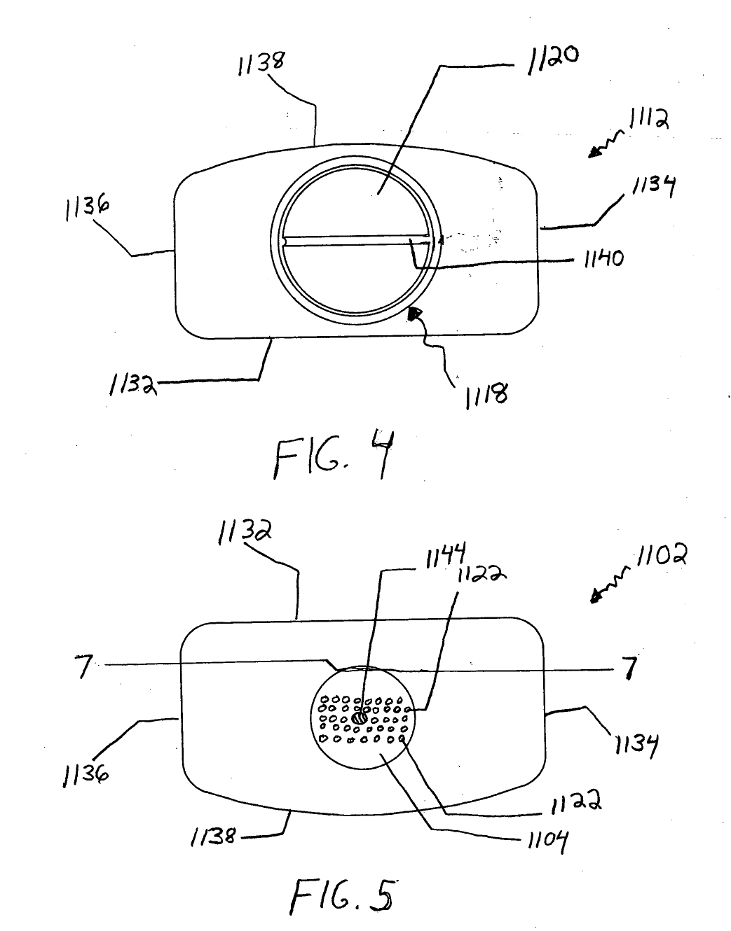

As illustrated in FIGS. 4 and 5, the members 1102, 1112 can be generally rectangular in shape. For example, the members can have a substantially straight posterior side 1132. A first substantially straight lateral side 1134 and a second substantially straight lateral side 1136 can extend substantially perpendicularly from the posterior side 1132 to an anterior side 1138. In a particular embodiment, the anterior side 1138 can curve outward such that the member is wider through the middle than along the lateral sides 1134, 1136. Further, in a particular embodiment, the lateral sides 1134, 1136 are substantially the same length.

FIG. 6 shows that the members 1102, 1112 can include at least one implant inserter engagement hole 1140. In a particular embodiment, the implant inserter engagement holes 1140 are configured to receive respective dowels, or pins, that extend from an implant inserter (not shown) that can be used to facilitate implantation of the prosthetic

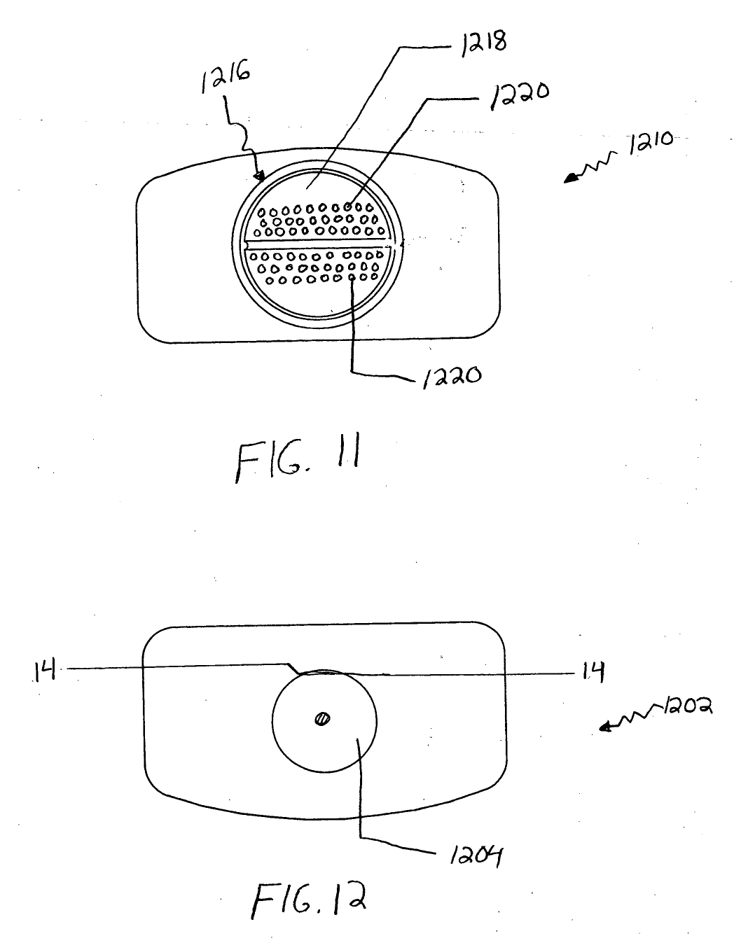

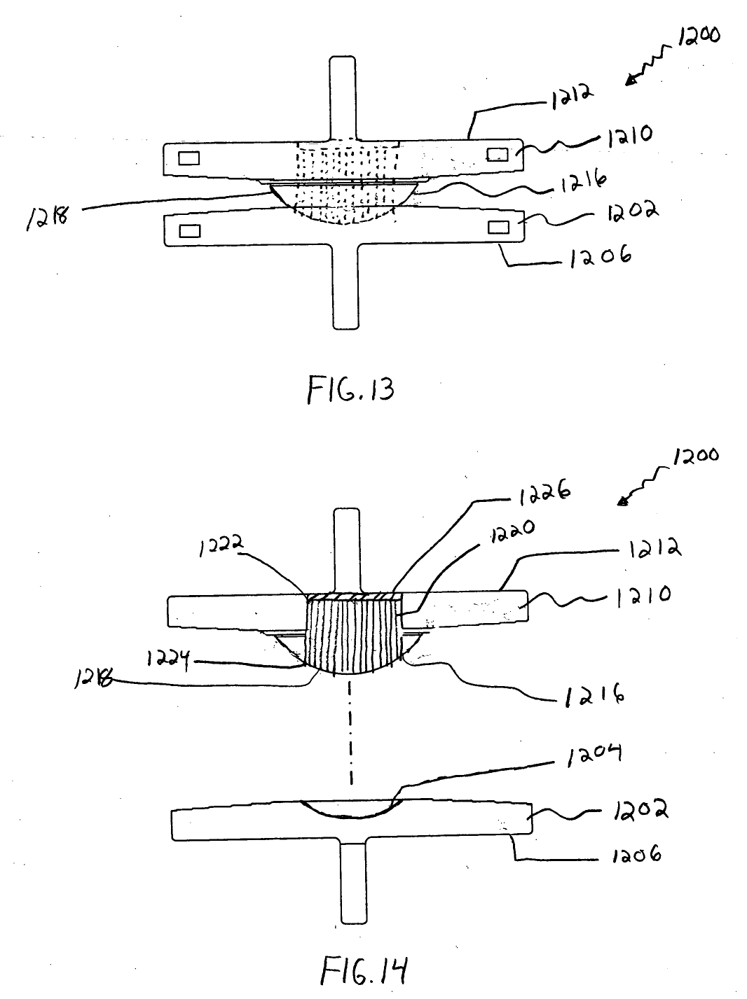

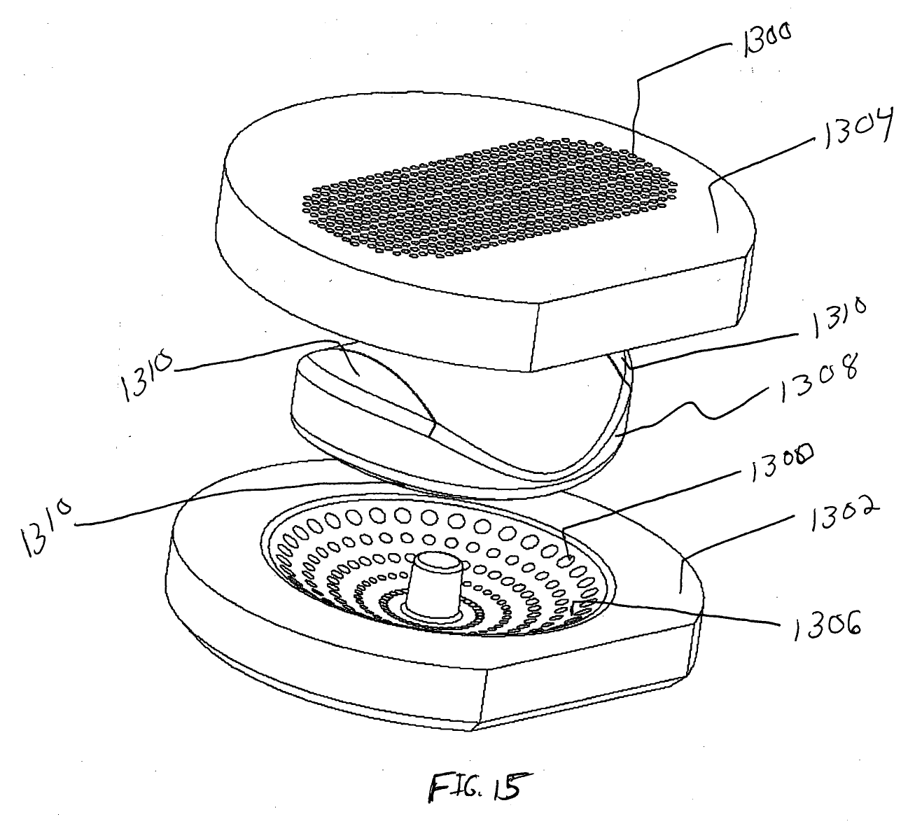

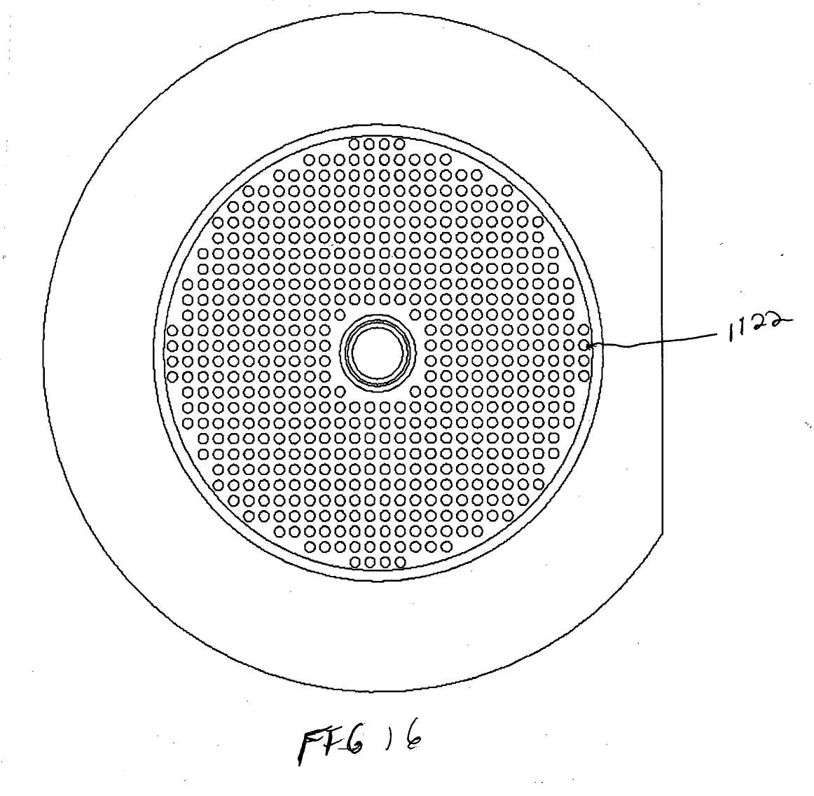

As shown in FIGS. 11-14, another exemplary embodiment is directed to an intervertebral prosthetic implant 1200 which can include a substantially rigid first member 1202 having an articulation surface 1204 and an engagement surface 1206 configured to engage a first vertebra and a substantially rigid second member 1210 having an engagement surface 1212 configured to engage a second vertebra. The second member 1210 can include an articulation member 1216 having an articulation surface 1218. The articulation member 1216 can be configured to facilitate motion between the first and second members 1202, 1210. The second member 1210 can have at least one fluid channel 1220 having a proximal end 1222 and a distal end 1224 relative to the engagement surface 1212 and configured to transport lubricious fluid to the articulation surface 1218 of the articulation member 1216. The fluid channels 1220 can be sized and shaped to act as capillary channels in transporting the lubricious fluid within the implant at least in part by capillary force. A semi-permeable membrane 1226 can be disposed proximate to the proximal end 1222 of at least one of the fluid channels 1220 or between the proximal end 1222 of at least one of the fluid channels 1220 and the articulation surface 1218 of the articulation member 1220.

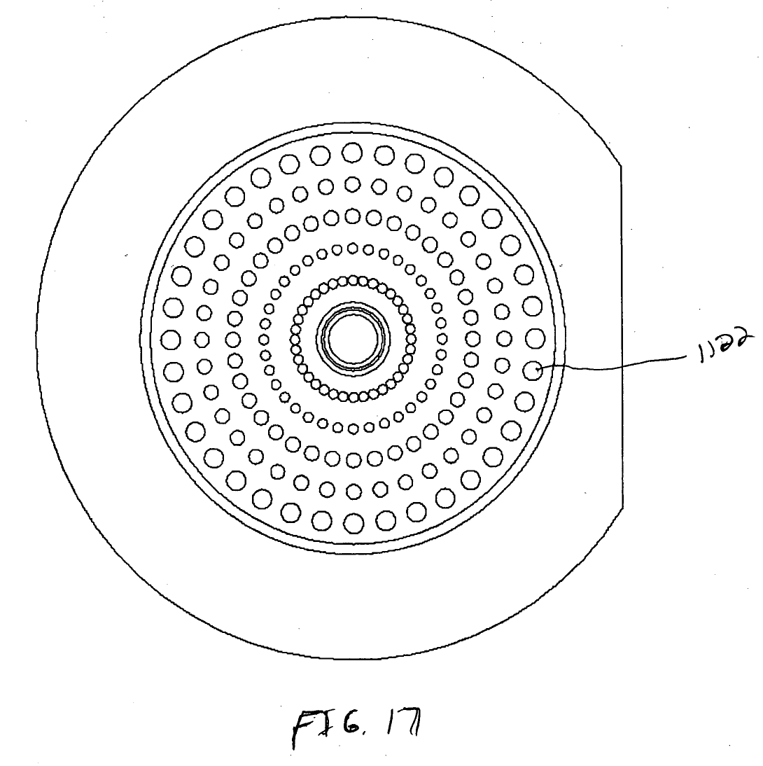

In various embodiments, the articulation member and the substantially rigid second member can be formed as a substantially monolithic structure. Alternatively, the articulation member can be at least partially attached to the second member or the articulation member can be at least partially attached to both members when motion restriction is desired. As shown in FIG. 15, further alternative embodiments can be configured such that at least one fluid channel 1300 is provided in both substantially rigid members 1302, 1304. One or more of the fluid channels 1300 can be sized and shaped to act as a capillary channel. Semi-permeable membranes (not shown) can be disposed proximate to the proximal ends of at least one of the fluid channels 1300 of both members 1302, 1304. Both the members 1302, 1304 can be provided with at least one articulation surface 1306. An articulation member 1308 can be provided with one or more additional articulation surfaces 1310 facing the articulation surfaces 1308 of the members 1302, 1304. The fluid channels and semi-permeable membrane can be placed in fluid communication with the additional articulation surfaces of the articulation member to provide lubricious fluid at these articulation surfaces, such that the articulation member is receiving lubricious fluid through each member.

Any of the substantially rigid components described herein can be formed of non-reactive polymers or biocompatible metals, alloys or ceramics. The polymers can include acrylonitrile polymers such as acrylonitrile-butadiene-styrene terpolymer, or the like; halogenated polymers such as polytetrafluoroethylene, polychlorotrifluoroethylene copolymer tetrafluoroethylene or hexafluoropropylene; polyimide; polysulfone; polycarbonate; polyethylene; polypropylene; polyvinylchloride-acrylic copolymer; polycarbonate-acrylonitrile-butadiene-styrene; polystyrene; as well as polyether materials such as polyetherketone (PEK), polyetheretherketone (PEEK), polyetherketoneketone (PEKK), and polyaryletherketone (PAEK), or the like. Exemplary metallic materials include stainless steel, titanium, platinum, tantalum, gold, and their alloys, as well as gold-plated ferrous alloys, platinum-plated ferrous alloys, cobalt-chromium alloys and titanium nitride coated stainless steel.

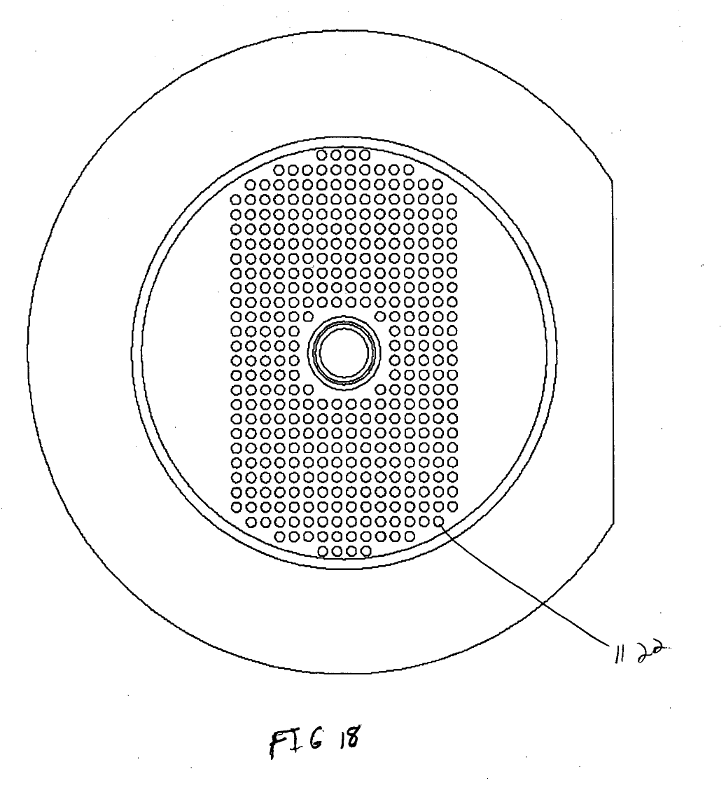

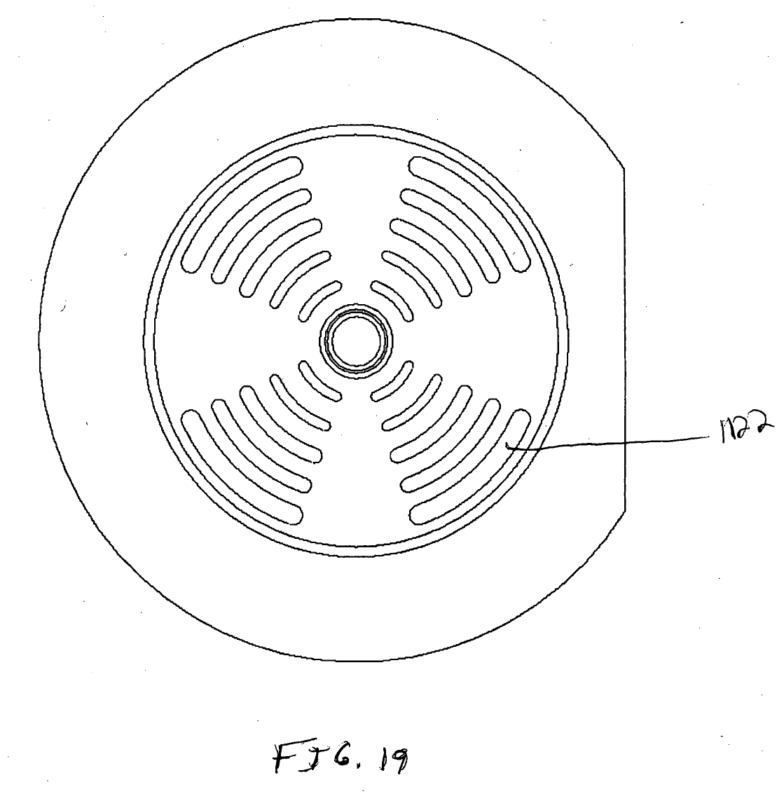

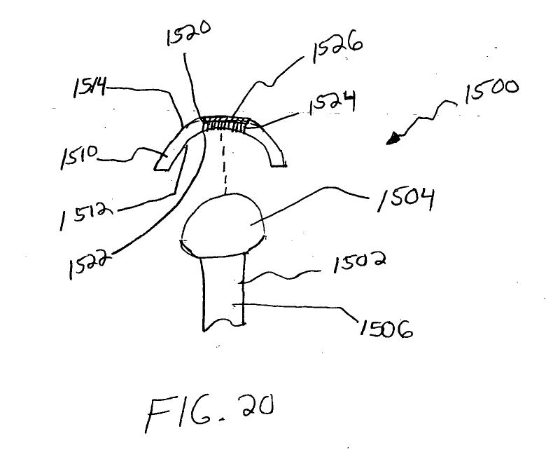

In further alternative embodiments, at least one of the first member or the second member has a plurality of fluid or capillary channels. As illustrated in FIGS. 16-19, the channels 1122 can be configured in any number of patterns, such as a radial pattern or a polygonal pattern. When desired, the channels can be concentrated near areas of anticipated wear in order to prioritize delivery of lubricious fluid to a high priority point of use. In certain embodiments, it may be desirable to concentrate the channels near the center of the articulation surface of one of the members.

As exemplified in FIGS. 16-19, the channels 1122 can be provided in various shapes. In exemplary embodiments, some of the channels can have a substantially circular cross-sectional shape. In other embodiments, some of the channels can have a substantially oblong cross-sectional shape. In further exemplary embodiments, some of the channels have a substantially polygonal cross-sectional shape or a substantially square cross-sectional shape. The channels can be provided in various lengths and thicknesses or diameters.

The size and shape of a channel can affect its capillarity. In certain embodiments it may be desirable to configure the fluid channels such that some or all of them function as capillary channels, which, for purposes of this disclosure means a passageway through a member which is sized and shaped to facilitate movement of a target fluid by capillary forces through the passageway. In certain embodiments, the channel can be configured such that capillary force transports the target fluid from a source near the proximal end to a semi-permeable membrane. In certain embodiments, the channel can be configured such that capillary force transports the target fluid from a source near the proximal end through the length of the channel to the distal end. In other embodiments, the channel can be configured such that capillary force assists in transporting the target fluid away from a semi-permeable membrane and toward a point of use near or at an articulation surface. For purposes of this Description, the proximal end of a channel is the end nearest the bone being engaged by the implant/joint or nearest the surface of the implant/joint configured to engage a bone.

In certain embodiments, the cross-sectional size of at least one of the channels can vary along the length of the channel. For example, the cross-sectional size of at least one of the channels can increase along the length of the channel from the proximal end to the distal end. This configuration allows fluid to move from a portion of the channel where capillarity is relatively high to a portion of the channel where capillarity is lower. This configuration can find utility in embodiments incorporating a semi-permeable membrane within the channel or proximate the proximal end of the channel. In certain embodiments, the portion of the channel from the proximal end to the semi-permeable membrane can be narrower and have a greater capillarity than the portion of the channel between the semi-permeable membrane and the distal end. This difference in capillarity can substantially reduce the capillary force acting on the fluid at or near the semi-permeable membrane in order to allow for a more even draw of fluid in embodiments having multiple fluid channels with an uneven distribution of source fluid near the proximal ends of the channels. Furthermore, certain embodiments can benefit from a larger channel on the solute side of the semi-permeable membrane in order to increase osmotic potential on the solute side of the membrane.

In embodiments having a semi-permeable membrane, a solute can be disposed between the semi-permeable membrane and the articulation surface of the articulation member. The solute can be present in sufficient concentration to provide an effective amount of the lubricious fluid at the articulation surface of the articulation member in vivo. In this context, the term “effective amount” means that amount which will exhibit a lubricating effect on at lest a portion of the articulation surface. In alternative embodiments, at least a portion of the solute can be contained in a timed release delivery system, such as in one or more tablets and/or capsules that dissolve or otherwise deteriorate over time, in order to produce an extended or staged release of solute on the solute side of the membrane. Various embodiments can include tablets and/or capsules of various dissolution rates. In certain embodiments, the staged delivery of solute maintains an effective amount of the lubricious fluid at the articulation surface of the articulation member over a period of at least one year. In other embodiments, the effective amount of the lubricious fluid is maintained at the articulation surface of the articulation member for a period of at least five years.

In various embodiments, a solute is chosen that has a molecular weight sufficiently high to retard permeation of the solute through the semi-permeable membrane such that the effective amount of the lubricious fluid is maintained at the articulation surface of the articulation member over a period of at least one year. In other embodiments, the effective amount of the lubricious fluid is maintained at the articulation surface of the articulation member for a period of at least five years.

Lubricious fluid is passed across the semi-permeable membrane by osmosis and is transported to and/or maintained at an articulation surface on the solute side of the membrane by osmotic potential and/or osmotic pressure. Osmosis is the passage of a solvent through a semi-permeable membrane separating two solutions of different concentrations. A semi-permeable membrane allows passage of solvents and is selectively permeable to various solutes. There is a tendency for the separated solutions to become the same concentration as the solvent passes from lower concentration to higher concentration. Osmosis will stop when the two solutions become equal in concentration or when pressure is applied to the solution containing higher concentration. When the higher concentrated solution is in a substantially closed system, that is when system is of substantially constant volume, there is a build up of pressure as the solvent passes from low to high concentration (i.e., osmotic pressure). Osmotic pressure can be calculated from the formula TT=nRT/V, where n/V denotes the concentration of the solution in mol/L, R is the gas constant and T denotes absolute temperature. Thus an approx. 1% solution of sodium chloride on the solute side (corresponding to a concentration of approx. 0.3 mol/L) will result in a relatively high osmotic pressure of 7 to 8 bar across the membrane.

In alternative embodiments, the cavity between the semi-permeable membrane and the relevant articulation surface can be filled with a desired solution prior to implantation or subsequently through a suitable valve in communication with the cavity. The solution can be delivered through the insertion device or with another device after the insertion device is removed. Solution can also be allowed to pass into the cavity after implantation.

The solute can be chosen from a number of liquid-attracting agents used to drive the flow of a lubricious solvent, such as water, synovial fluid or the like. The solute may be an osmagent, an osmopolymer, or a mixture of the two. An osmagents is a nonvolatile species which is soluble in lubricious fluid and creates an osmotic potential which drives the osmotic inflow of lubricious fluid. A non-exclusive, exemplary listing of osmagents include magnesium sulfate, magnesium chloride, potassium sulfate, sodium chloride, sodium sulfate, lithium sulfate, sodium phosphate, potassium phosphate, d-mannitol, sorbitol, inositol, urea, magnesium succinate, tartaric acid, raffinose, and various monosaccharides, oligosaccharides and polysaccharides such as sucrose, glucose, lactose, fructose, and dextran, as well as mixtures of any of these various species. The solute may additionally contain a contrasting agent to make the implant opaque for X-rays. In certain embodiments, the contrasting agent can be introduced in a liquid state.

Osmopolymers are generally hydrophilic polymers that can swell upon contact with lubricious fluid and can be of plant or animal origin, or may be synthetic. A non-exclusive exemplary listing of osmopolymers include: poly(hydroxy-alkyl methacrylates) with a molecular weight of 30,000 to 5,000,000, poly(vinylpyrolidone) with a molecular weight of 10,000 to 360,000, anionic and cationic hydrogels, polyelectrolyte complexes, poly(vinyl alcohol) having low acetate residual—optionally cross-linked with glyoxal, formaldehyde or glutaraldehyde and having a degree of polymerization of 200 to 30,000, mixtures of methyl cellulose, cross-linked agar and carboxymethylcellulose, mixtures of hydroxypropyl methylcellulose and sodium carboxymethylcellulose, polymers of N-vinyllactams, polyoxyethylene-polyoxypropylene gels, polyoxybutylene-polyethylene block copolymer gels, carob gum, polyacrylic gels, polyester, gels, polyurea gels, polyether gels, polyamide gels, polypeptide gels, polyamino acid gels, polycellulosic gels, carbopol acidic carboxy polymers having molecular weights of 250,000 to 4,000,000, Cyanamer polyacrylamides, cross-linked indene-maleic anhydride polymers, polyacrylic acids having molecular weights of 80,000 to 200,000, starch graft copolymers, and acrylate polymer polysaccharides.

The semi-permeable membrane can be formed of a semi permeable material which allows passage of lubricious fluids, especially water, while limiting the passage of solutes. A non-exclusive listing of semi permeable materials include polyester elastomers, cellulose esters, cellulose ethers and cellulose ester-ethers, water flux enhanced ethylene-vinyl acetate copolymers, silicone, polyurethane, polycarbonate-urethane, silicone-polycarbonate-urethane or silicone-polyetherurethane. Any of the above can be provided with a coating or admixed with a material that reduces hydrophobic characteristics. The cellulosic polymers listed above can have a degree of substitution, D.S., on the anhydroglucose unit, from greater than 0 up to 3 inclusive. In this context, “D.S.” means the average number of hydroxyl groups originally present on the anhydroglucose unit comprising the cellulose polymer that are replaced by a substituting group. Representative materials include cellulose acylate, cellulose diacetate, cellulose triacetate, mono-, di-, and tricellulose alkanylates, mono-, di-, and tricellulose aroylates, and the like. Exemplary cellulosic polymers include cellulose acetate having a D.S. up to 1 and an acetyl content up to 21%; cellulose acetate having a D.S. of 1 to 2 and an acetyl content of 21% to 35%; cellulose acetate having a D.S. of 2 to 3 and an acetyl content of 35% to 44.8%, and the like. More specific cellulosic polymers include cellulose propionate having a D.S. of 1.8 and a propionyl content of 39.2% to 45% and a hydroxyl content of 2.8% to 5.4%; cellulose acetate butyrate having a D.S. of 1.8 and an acetyl content of 13% to 15% and a butyryl content of 34% to 39%; cellulose acetate butyrate having an acetyl content of 2% to 29%, a butyryl content of 17% to 53% and a hydroxyl content of 0.5% to 4.7%; cellulose acetate butyrate having a D.S. of 1.8, and acetyl content of 4% average weight percent and a butyryl content of 51%; cellulose triacylates having a D.S. of 2.9 to 3 such as cellulose trivalerate, cellulose trilaurate, cellulose tripalmitate, cellulose trisuccinate, and cellulose trioctanoate; cellulose diacylates having a D.S. of 2.2 to 2.6 such as cellulose disuccinate, cellulose dipalmitate, cellulose dioctanoate, cellulose dipentate; coesters of cellulose such as cellulose acetate butyrate and cellulose, cellulose acetate propionate, and the like. Any of the semi-permeable materials can be mixed with barium sulfate or the like to make them opaque for X-rays.

The concepts and features described herein also find utility in other prosthetic joints, such as hip and/or knee prostheses. Accordingly, another embodiment is directed to a prosthetic hip joint 1500, as shown in FIG. 20, which can have a substantially rigid first member 1502 with an articulation surface 1504 and an engagement surface 1506 configured to engage a first bone. The prosthetic joint can also include a substantially rigid second member 1510 having an articulation surface 1512 and an engagement surface 1514 configured to engage a second bone. At least one of the first or second members 1502, 1510 can have at least one fluid channel 1524 configured to transport lubricious fluid to at least one of the articulation surfaces 1504, 1518.

In alternative embodiments, the fluid channels can be configured to transport lubricious fluid at least in part by capillary force. Further, at least one of the first or second members 1502, 1510 can have a plurality of capillary channels. At least one of the capillary channels can vary in cross-sectional size along its length, such as by increasing in cross-sectional size along its length from a proximal end 1520 to a distal end 1522. In various alternative embodiments, a semi-permeable membrane 1526 can be disposed proximate to the proximal end 1520 of at least one fluid channel 1524 or at any functional location between the proximal end 1520 and the articulation surface. As will be appreciated by the skilled practitioner, the substantially rigid members 1502, 1510 can be monolithic or can comprise multiple components as dictated by the situation.

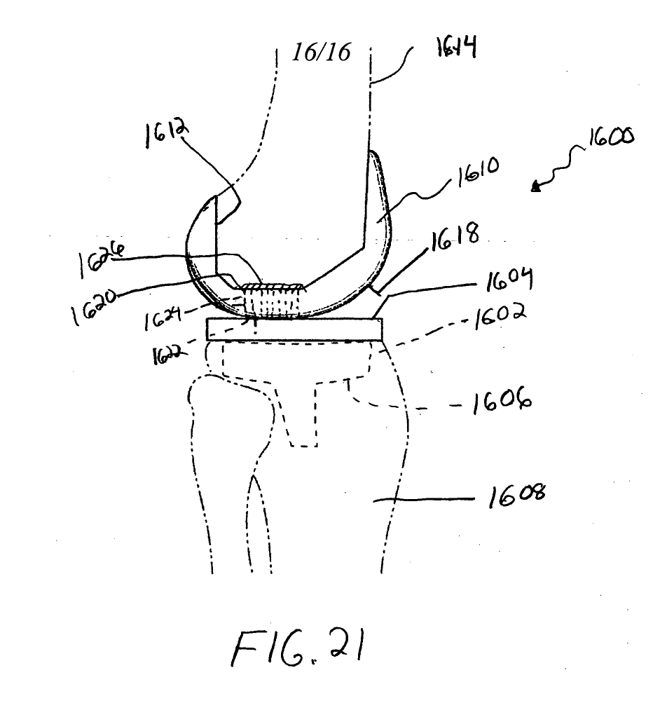

Another embodiment is directed to a prosthetic knee joint 1600, as shown in FIG. 21, which can have a substantially rigid first member 1602 with an articulation surface 1604 and an engagement surface 1606 configured to engage a first bone 1608. The prosthetic joint can also include a substantially rigid second member 1610 having an articulation surface 1618 and an engagement surface 1612 configured to engage a second bone 1614. At least one of the first or second members 1602, 1610 can have at least one fluid channel 1624 configured to transport lubricious fluid to at least one of the articulation surfaces 1604, 1618.

In alternative embodiments, the fluid channels can be configured to transport lubricious fluid at least in part by capillary force. Further, at least one of the first or second members 1602, 1610 can have a plurality of capillary channels. At least one of the capillary channels can vary in cross-sectional size along its length, such as by increasing in cross-sectional size along its length from a proximal end 1620 to a distal end 1622. In various alternative embodiments, a semi-permeable membrane 1626 can be disposed proximate to the proximal end 1620 of at least one fluid channel 1624 or at any functional location between the proximal end 1620 and the articulation surface. As will be appreciated by the skilled practitioner, the substantially rigid members 1602, 1610 can be monolithic or can comprise multiple components as dictated by the situation.

The prosthetic devices described herein can be implanted in any art-recognized method according to relevant indications and preferences of the surgeon. When the device is an intervertebral prosthetic implant, the method can include gaining access to the problematic disc and performing a discectomy to remove at least a portion of the disc thereby creating an intervertebral space for receiving the implant. One or more keel grooves may be cut into the cortical rim and cancellous bone to receive keel(s) if such is provided on the first and second members.

After installation, the members can be in direct contact with vertebral bone, e.g., cortical bone and cancellous bone. Consequently, the bone-contacting surface of the members can be coated with a bone-growth promoting substance, e.g., a hydroxyapatite coating formed of calcium phosphate. Additionally, the contact surface can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

It will be understood that each of the elements described above, or two or more together, may also find utility in applications differing from the types described herein. While the invention has been illustrated and described as embodied in a passive lubricating prosthetic joint, it is not intended to be limited to the details shown, since various modifications and substitutions can be made without departing in any way from the spirit of the present invention. For example, multi-component end plates can be employed with the intervertebral embodiments of the present prosthetic when desired. Further, although many examples of various alternative biocompatible chemicals and materials have been presented throughout this specification, the omission of a possible item is not intended to specifically exclude its use in or in connection with the claimed invention. As such, further modifications and equivalents of the invention herein disclosed may occur to persons skilled in the art using no more than routine experimentation, and all such modifications and equivalents are believed to be within the spirit and scope of the invention as defined by the following claims.

1. An intervertebral prosthetic implant, comprising:
a substantially rigid first member having an articulation surface and an engagement surface configured to engage a first vertebra;
a substantially rigid second member having an engagement surface configured to engage a second vertebra; and
an articulation member having an articulation surface and configured to facilitate motion between the first and second members, wherein the first member has a capillary channel having a proximal end and a distal end relative to the engagement surface of the first member and configured to transport lubricious fluid proximate the articulation surface of the articulation member at least in part by capillary force.

2. The intervertebral prosthetic implant of claim 1, wherein the articulation member and the substantially rigid second member are a substantially monolithic structure.

3. The intervertebral prosthetic implant of claim 1, wherein the articulation member has a plurality of articulation surfaces.

4. The intervertebral prosthetic implant of claim 3, wherein the second member has an articulation surface.

5. The intervertebral prosthetic implant of claim 1, wherein the second member has a capillary channel having a proximal end and a distal end relative to the engagement surface of the second member and configured to transport lubricious fluid proximate the articulation surface of the articulation member.

6. The intervertebral prosthetic implant of claim 1, wherein at least one of the first member or the second member has a plurality of capillary channels.

7. The intervertebral prosthetic implant of claim 6, wherein a portion of the capillary channels are configured in a radial pattern.

8. The intervertebral prosthetic implant of claim 6, wherein a portion of the capillary channels are configured in a polygonal pattern.

9. The intervertebral prosthetic implant of claim 6, wherein the capillary channels are concentrated near areas of anticipated wear on the articulation surface of the articulation member.

10. The intervertebral prosthetic implant of claim 6, wherein the capillary channels of the first member are concentrated near the center of the articulation surface of the first member.

11. The intervertebral prosthetic implant of claim 6, wherein a portion of the capillary channels have a substantially circular cross-sectional shape.

12. The intervertebral prosthetic implant of claim 6, wherein a portion of the capillary channels have a substantially oblong cross-sectional shape.

13. The intervertebral prosthetic implant of claim 6, wherein a portion of the capillary channels have a substantially polygonal cross-sectional shape.

14. The intervertebral prosthetic implant of claim 1, wherein the cross-sectional size of the capillary channel varies along the length of the channel.

15. The intervertebral prosthetic implant of claim 14, wherein the cross-sectional size of the capillary channel increases along the length of the channel from the proximal end to the distal end.

16. The intervertebral prosthetic implant of claim 1, further comprising a semi-permeable membrane disposed proximate the proximal end of the capillary channel of the first member.

17. The intervertebral prosthetic implant of claim 5, further comprising a semi-permeable membrane disposed proximate the proximal end of the capillary channel of the second member.

18. The intervertebral prosthetic implant of claim 5, wherein the articulation member comprises a capillary channel in fluid communication with the capillary channel of the second member.

19. An intervertebral prosthetic implant, comprising:
a substantially rigid first member having an articulation surface and an engagement surface configured to engage a first vertebra;
a substantially rigid second member having an engagement surface configured to engage a second vertebra;
an articulation member having an articulation surface and configured to facilitate motion between said first and second members, wherein the first member has a fluid channel having a proximal end and a distal end relative to the engagement surface of the first member and configured to allow lubricious fluid to flow proximate the articulation surface of the articulation member; and
a semi-permeable membrane disposed proximate the proximal end of the fluid channel.

20. The intervertebral prosthetic implant of claim 19, further comprising a solute disposed between the semi-permeable membrane and the articulation surface of the articulation member, the solute being present in sufficient concentration to provide an effective amount of the lubricious fluid at the articulation surface of the articulation member in vivo.

21. The intervertebral prosthetic implant of claim 20 wherein at least a portion of the solute is contained in a timed release delivery system.

22. The intervertebral prosthetic implant of claim 20, wherein the solute has a molecular weight that retards permeation of the solute through the semi-permeable membrane such that the effective amount of the lubricious fluid is maintained at the articulation surface of the articulation member over a period of at least one year.

23. The intervertebral prosthetic implant of claim 22, wherein the solute has a molecular weight that retards permeation of the solute through the semi-permeable membrane such that the effective amount of the lubricious fluid is maintained at the articulation surface of the articulation member over a period of at least five years.

24.-34. (canceled)

35. An intervertebral prosthetic implant, comprising:
a substantially rigid first member having an articulation surface and an engagement surface configured to engage a first vertebra and
a substantially rigid second member having an engagement surface configured to engage a second vertebra, the second member comprising an articulation member having an articulation surface and configured to facilitate motion between the first and second members, wherein the second member has a capillary channel having a proximal end and a distal end relative to the engagement surface and configured to transport lubricious fluid to the articulation surface of the articulation member at least in part by capillary force.

36. An intervertebral prosthetic implant, comprising:
a substantially rigid first member having an articulation surface and an engagement surface configured to engage a first vertebra;
a substantially rigid second member having an engagement surface configured to engage a second vertebra, the second member comprising an articulation member having an articulation surface and configured to facilitate motion between the first and second members, wherein the second member has a fluid channel having a proximal end and a distal end relative to the engagement surface and configured to allow lubricious fluid to flow proximate the articulation surface of the articulation member; and
a semi-permeable membrane disposed proximate to the proximal end of the fluid channel.

37. A prosthetic joint, comprising:
a substantially rigid first member having an articulation surface and an engagement surface configured to engage a first bone and
a substantially rigid second member having an articulation surface and an engagement surface configured to engage a second bone,
wherein at least one of the first member or the second member has a capillary channel having a proximal end and a distal end relative to the engagement surface and configured to transport lubricious fluid to the articulation surface of at least one of the first member or the second member at least in part by capillary force.

38.-40. (canceled)

41. A prosthetic joint, comprising:
a substantially rigid first member having an articulation surface and an engagement surface configured to engage a first bone;
a substantially rigid second member having an articulation surface and an engagement surface configured to engage a second bone, wherein at least one of the first member or the second member has a fluid channel having a proximal end and a distal end relative to the engagement surface and configured to allow lubricious fluid to flow proximate the articulation surface of at least one of the first member or the second member; and
a semi-permeable membrane disposed proximate to the proximal end of the fluid channel of the first member or the second member.

42.-44. (canceled)

45. A method of replacing at least a portion of an intervertebral disc, the method comprising the following steps:
A. Gaining access to the intervertebral disc;
B. Removing at least a portion of the intervertebral disc to create an intervertebral space; and
C. Inserting a prosthetic disc into the intervertebral space, the prosthetic disc comprising a substantially rigid first member having an articulation surface and an engagement surface configured to engage a first vertebra;
a substantially rigid second member having an engagement surface configured to engage a second vertebra; and
an articulation member having an articulation surface and configured to facilitate motion between the first and second members, wherein at least one of the first member or the second member has a capillary channel having a proximal end and a distal end relative to the engagement surface and configured to transport lubricious fluid to the articulation surface of the articulation member at least in part by capillary force.

46. A method of replacing at least a portion of an intervertebral disc, the method comprising the following steps:
A. Gaining access to the intervertebral disc;
B. Removing at least a portion of the intervertebral disc to create an intervertebral space; and
C. Inserting a prosthetic disc into the intervertebral space, the prosthetic disc comprising a substantially rigid first member having an articulation surface and an engagement surface configured to engage a first vertebra;
a substantially rigid second member having an engagement surface configured to engage a second vertebra;
an articulation member having an articulation surface and configured to facilitate motion between the first and second members, wherein at least one of the first member or the second member has a fluid channel having a proximal end and a distal end relative to the engagement surface and configured to allow lubricious fluid to flow proximate the articulation surface of the articulation member; and
a semi-permeable membrane disposed proximate to the proximal end of the fluid channel of the first member or the second member.

47.-49. (canceled)

50. An intervertebral prosthetic implant, comprising:
a substantially rigid first member having an articulation surface and an engagement surface configured to engage a first vertebra;
a substantially rigid second member having an engagement surface configured to engage a second vertebra;
an articulation member having an articulation surface and configured to facilitate motion between said first and second members, wherein the first member has a fluid channel having a proximal end and a distal end relative to the engagement surface of the first member and configured to allow lubricious fluid to flow proximate the articulation surface of the articulation member; and
a semi-permeable membrane disposed between the proximal end of the fluid channel and the articulation surface of the articulation member.
